(19) United States
(12) Patent Application Publication
(10) Pub. No.: US 2021/0169548 A1
(43) Pub. Date: Jun. 10, 2021

(54) Instrument for Electrosurgical Treatment

(71) Applicant: Erbe Elektromedizin GmbH, Tuebingen (DE)

(72) Inventors: Sandra Keller, Hechingen (DE); Markus Enderle, Tuebingen (DE); Michael Ederer, Stuttgart (DE)

(21) Appl. No.: 17/112,515

(22) Filed: Dec. 4, 2020

(30) Foreign Application Priority Data
Dec. 5, 2019 (EP) 19213828.7

(51) Int. Cl. A61B 18/04; A61B 1/018; A61B 90/92; H05H 1/46

(52) U.S. Cl. CPC A61B 18/042; A61B 1/018; A61B 2018/00982; H05H 1/46; A61B 90/92

(57) ABSTRACT

An instrument for thermal treatment of tissue (34), particularly for electrosurgical treatment and particularly for argon plasma coagulation, is disclosed. The instrument has an instrument body (25) extending between a proximal end (27) and a distal end (28). In an end section (38) adjoining a distal end (28) the instrument body (25) comprises at least one color mark (39) in a defined color. This color corresponds to the color of a treated tissue (34) that is created, if the dosage of an energy introduction for achieving a desired depth effect in the tissue (34) has been selected correctly. Preferably the instrument is configured as probe (17), particularly as endoscope probe and has a flexible bendable instrument body (25) that can be referenced as instrument hose (26).

### RELATED APPLICATION(S)

This application claims the benefit of European Patent Application No. 19213828.7, filed Dec. 5, 2019, the contents of which are incorporated herein by reference as if fully rewritten herein.

### TECHNICAL FIELD

The invention refers to an instrument, particularly a probe or endoscopic probe that is configured for thermal treatment, e.g. electrosurgical treatment of biological tissue. The instrument or probe can thermally influence the biological tissue by means of a rigid body or also by means of a plasma. For example, the instrument can be configured for cauterization or for plasma coagulation, particularly argon plasma coagulation.

### BACKGROUND

Such an instrument is known from DE 198 20 240 A1, for example. The instrument can be guided through an operation channel of an endoscope. Adjoining the distal end the instrument comprises marking rings by means of which the surgeon is able to recognize how far the distal end projects out of the endoscope.

During the thermal and particularly electrosurgical treatment of tissue, the dosage of the treatment is frequently difficult to estimate for the surgeon, i.e. how long the biological tissue has to be thermally treated in the application area or what amount of energy per unit area has to be introduced into the biological tissue in order to achieve the desired depth effect. The tissue must neither be treated too short, nor too long in order to avoid underdosage or overdosage and in order to achieve the desired effect, particularly the desired depth effect.

Starting herefrom it is an object of the present invention to provide a probe that facilitates the dosing, particularly the estimation of the duration of the treatment in the application area.

### SUMMARY

This object is solved by a probe as disclosed herein.

The instrument or the probe is configured for thermal treatment of biological tissue. The tissue is treated in an application area, particularly by means of a plasma. Preferably the instrument or the probe is configured for electrosurgical coagulation, particularly for argon plasma coagulation.

The instrument comprises an instrument body that can be formed by a rigid tube body that is non-bendable by the forces that regularly occur or by a flexible hose body. The instrument body extends from a proximal end to a distal end. The instrument body can be connected to a supply and operating unit at the proximal end.

The instrument comprises an electrode in the area of the distal end of the instrument body to which a voltage, particularly a radio frequency AC-voltage can be applied. The electrode can be connected with an electric terminal by means of an electric conductor extending in the instrument body, wherein the electric terminal can be particularly arranged on the proximal end of the instrument or the probe. A supply line of a supply and operating unit can be connected with the electric terminal in order to be able to provide the voltage to the electrode.

A thermal influence of the biological tissue can be carried out by means of the electrode by direct contact (e.g. during cauterization) or indirectly via an electric conductible medium (e.g. during coagulation).

In an embodiment the instrument or the probe is configured for plasma coagulation, particularly for argon plasma coagulation. Then a fluid channel can be formed in the instrument body that is fluidically connected with an exit opening in the area of the distal end. Preferably the electrode is arranged in the area of the exit opening inside the instrument body. The electrode is flushed by a fluid, particularly an inert gas, a gas mixture or an additive added to the inert gas.

In this configuration of the instrument in a preferred application the fluid exiting the exit opening, particularly the inert gas, can be ionized and can be brought in an electric conductible state of aggregation in which it is transferred into a plasma. The fluid is directed on the biological tissue in the application area. Due to the electric conductible state of aggregation of the effusing ionized fluid, energy is transferred to the biological tissue. For the ionization of the fluid, particularly the argon gas, a radio frequency AC-voltage is applied to the electrode that can be supplied to the instrument via the supply and operating unit. The ionization of the inert gas occurs in the area directly in front of the exit opening.

In an application the energy conducted via the electrode is transferred to the tissue in the application area, e.g. by means of contact of the electrode to the tissue. The energy transferred to the electrode is realized by a radio frequency AC-voltage that can be supplied to the instrument via the supply and operating unit.

At least one color mark is provided on the distal end of the instrument body that has a defined color or a defined color shade and/or a defined color saturation and/or a defined brightness, wherein the color can be, for example, brown, brown-beige or red. The at least one color mark is arranged to be visible from outside and can comprise, for example, a colored print and/or a colored insert. In addition or as an alternative, part of the instrument body can be manufactured in the selected or defined color and/or can be at least partly colored or printed, such as for example an end piece comprising the exit opening. The at least one color mark can be preferably provided on a shell surface of the instrument body.

The at least one color mark can comprise at least one symbol and/or at least one sign (digit and/or letter) and/or at least one geometric figure, such as for example a logo and/or a barcode. For example, at least one colored ring can serve as color mark. The color mark or at least one of the color marks is preferably a continuous completely filled area in the desired or defined color.

The color of the color mark is selected such that it corresponds to the color of tissue treated with the desired dosage and/or depth effect. That is, the color mark serves a color comparison with the coloring of the treated tissue and the coloring of the treated tissue is most similar to the color mark, if the desired dosage or depth effect has been achieved. A surgeon can thus directly recognize by means of color comparison between the color mark and treated tissue whether the dosage or influence duration has been selected correctly and thus can conclude on the achieved depth effect. The following applies for a typical application: If the color of the treated tissue is brighter than the color of the color mark, the treatment was not carried out long enough. If the color of the treated tissue is darker than the color of the color mark, the treatment has been overdosed, particularly during a too long treatment of the tissue. If the color of the treated tissue and the color mark are equal, the correct dosage and treatment duration has been carried out and the desired depth effect has been achieved in the biological tissue. In other applications the color change can also be different from the described typical application. In any case, the at least one color mark indicates the color at which the desired dosage or depth effect has been achieved in the biological tissue in the respective application.

The color of the color mark is preferably brown or brown-beige. The color, the color shade or another color characteristic can be selected, particularly depending on the kind of treatment and on the type of tissue to be treated. In an application the color of the at least one color mark is an RAL-color. Depending on the clinical indication and the treatment area or type of tissue, RAL-colors with the RAL-numbers 1001, 1005, 1011, 1024, 8001, 8003, 8007 or 8011 can be used for the at least one color mark. It has shown that the RAL-colors indicate the color shades of the tissue treated with the correct dosage very well, wherein one of the RAL-colors is selected depending on the kind of treatment and on the type of tissue to be treated. In addition, the RAL-color is precisely defined and can be reproduced during the manufacturing of probes very well.

In order to allow a sufficiently exact comparison for the surgeon, it is advantageous if the color mark is sufficiently large. Preferably the color mark is two-dimensional and comprises preferably a total area of at least 15 mm² or at least 20 mm². In addition or as an alternative, the color mark can have a length in extension direction of the instrument body of at least 2 mm or at least 3 mm or at least 5 mm. It is further preferred, if the indicated total area of the color mark is configured as continuous area and is preferably completely filled with a defined color.

In a preferred embodiment the color mark or at least one of multiple color marks is provided at a shell surface of the instrument body, particularly in the form of a print. Here this color mark can have the shape of at least one stripe or ring.

In a preferred embodiment the color mark or the at least one of multiple color marks is provided at a shell surface of the instrument body, particularly in the form of at least a partly surrounding area, as e.g. a surrounding ring and/or two or more stripes. It is advantageous, if this at least one color mark extends in a circumferential direction around the instrument body at least about 50% of the circumference of the instrument body. Preferably the at least one color mark extends completely around the circumference of the instrument body and has the shape of a closed ring area.

Outside of the at least one color mark the instrument body has at least a color or multiple colors that distinguish from the color of the at least one color mark and that is particularly in optical contrast to the tissue. In doing so, the visibility of the instrument or the probe is improved for the surgeon. For example, the instrument body can be blue outside of or surrounding the at least one color mark. The color of the instrument body surrounding the at least one color mark is preferably darker than the color of the at least one color mark.

It is preferred that the at least one color mark does not extend up to the distal end of the instrument body, but is arranged with distance thereto.

It is also preferred that the at least one color mark is a mark that indicates a defined distance or minimum distance to the distal end. In doing so, it can be indicated to the surgeon when the instrument body or the distal end has been shifted far enough out of the endoscope to not damage or affect the optics of the endoscope during application of the plasma. For example, the edge of the color mark that is furthest away can define a minimum distance to the distal end. Only if at least one color mark can be completely seen through optics of the endoscope, the distal end of the instrument body is far enough away from the endoscope in order to execute a treatment.

In an embodiment the instrument body comprises an end piece that comprises the exit opening. For example, the end piece can be configured to define the effusing direction of the fluid or the plasma. The end piece can be completely made or partly made in the defined color of the color mark, so that the end piece forms at least one of the color marks.

The at least one color mark or at least on of multiple color marks can comprise a texture and/or surface structure and/or frosted and/or non-reflective surface. In doing so, the real tissue structure can be better depicted and the comparison between the textured color marking and the color and/or structure of the tissue can be simplified. In addition, a rough and/or frosted and/or non-reflective surface of the color mark can avoid light reflections. Thereby the surface can have preferably a roughness that corresponds to one of the VDI-classes 33-42 of the VDI-guideline 3400 (surface reference sample measurement). The surface can have a maximum depth of roughness Rmax=18-49 μm, particularly Rmax=25 μm and/or an arithmetic average roughness value Ra=4.5-12.5 μm, particularly Ra=6.3 μm.

The configurations of the at least one color mark discussed above can be arbitrarily combined with each other. For example, an end piece of the instrument body can serve as color mark, as well as one or more additional colored areas, symbols, signs or the like can be provided at the shell surface of the instrument body in addition. In a preferred embodiment the at least one color mark is exclusively provided on the outside of the shell surface of the instrument body. In this case, the instrument body can be used independent from the type and configuration of the exit opening during manufacturing of probes as non-variable part. In doing so, scaling effects can be achieved.

In an embodiment the instrument body is configured as instrument hose. The instrument hose is flexible or bendable transverse to its extension direction, due to the occurring forces. Preferably the instrument hose is configured to be guided through an operating channel of an endoscope.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. Preferred embodiments of the invention are apparent from the dependent claims, the description and the drawings. Subsequently, preferred embodiments of the invention are explained in detail with reference to the attached drawings. The drawings show:

FIG. 1 a schematic principle illustration of an endoscope as well as an instrument forming a probe that is guided through an operating channel of the endoscope,

FIG. 2 an end section of the probe of FIG. 1 projecting out of the operating channel of the endoscope in a schematic block diagram-like illustration,

FIGS. 3-5 an embodiment of exit openings of a probe for a plasma or a fluid in different configurations in each case in a schematic side view,

FIG. 6a a schematic illustration of a modified embodiment of the end section of a probe,

FIG. 6b a schematic illustration of a modified embodiment of the end section having a color mark that extends along the end section of a probe,

FIGS. 7-9 a schematic principle illustration of a color mark of a probe in comparison to the color of an application area of treated biological tissue,

FIG. 10 is a photographic illustration of an end section of a probe having a color mark and multiple application areas of a tissue with different application dosages of the probe and

FIGS. 11 and 12 further embodiments of a probe in each case in a schematic partial illustration.

### DETAILED DESCRIPTION

The invention refers to an instrument for thermal and for example, electrosurgical treatment of biological tissue 34. In the following embodiments illustrated in the drawings the instrument is configured as probe 17 that is preferably configured to be used in combination with an endoscope 15.

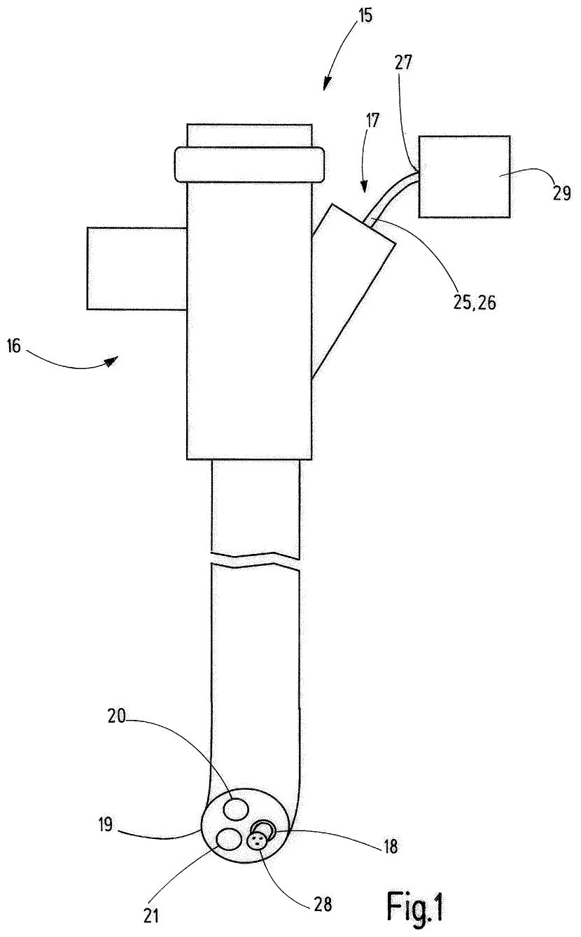

FIG. 1 shows the endoscope 15 in the manner of a block diagram. The endoscope 15 comprises an operating part 16 that can be handled by a surgeon. The probe can be inserted into an operation channel 18 of the endoscope 15 at the operating part 16. The operation channel 18 has a port at the endoscope end 19 opposite the operating part 16, out of which at least a section of the probe 17 can be extended. Adjoining the port of the operation channel 18 a light source 20 and an objective lens 21 are arranged at the distal endoscope end 19. By means of the light source 20 the environment in front of the endoscope end 19 and particularly the tissue to be treated is illuminated. By means of the objective lens 21 and optics or camera an image of the environment in front of the endoscope end 19 and particularly the tissue to be treated is detected. The camera and/or the optics of the endoscope 15 forwards the images to an ocular and/or an external monitor.

The instrument configured as probe 17 has an instrument body 25 that is configured as instrument hose 26 in the embodiment that can be bent transverse to its extension direction by the occurring forces. Because the instrument is a probe 17, the instrument body 25 could also be referenced as probe body and the instrument hose could also be referenced as probe hose. The instrument body 25 and according to the example, the instrument hose 26 extends from a proximal end 27 to a distal end 28. At the proximal end 27 a supply and operating unit 29 is connected with the instrument hose 26.

In the illustrated embodiments the instrument and according to the example, the probe 17 is configured for plasma coagulation and according to the example, for argon plasma coagulation. Alternatively, the instrument could also be configured for cauterization or for carrying out of another thermal treatment of biological tissue.

The probe 17 comprises an electrode 32 in the area of the distal end 28 of the instrument body 25. The electrode 32 can be configured, for example, to directly contact the biological tissue 34 to be treated. In the preferred embodiment the electrode 32 is configured to carry out a thermal influence of the biological tissue 34 indirectly via an electric conductible medium.

A voltage, particularly a radio frequency AC-voltage, can be applied to the electrode 32. The electrode 32 is connected with an electric terminal via a not illustrated electric conductor extending in the instrument body 25, wherein the electric terminal is particularly arranged at the proximal end of the instrument or the probe. An electric connection can be established to the supply and operating unit 29 via the electric terminal in order to be able to apply the voltage to the electrode 32.

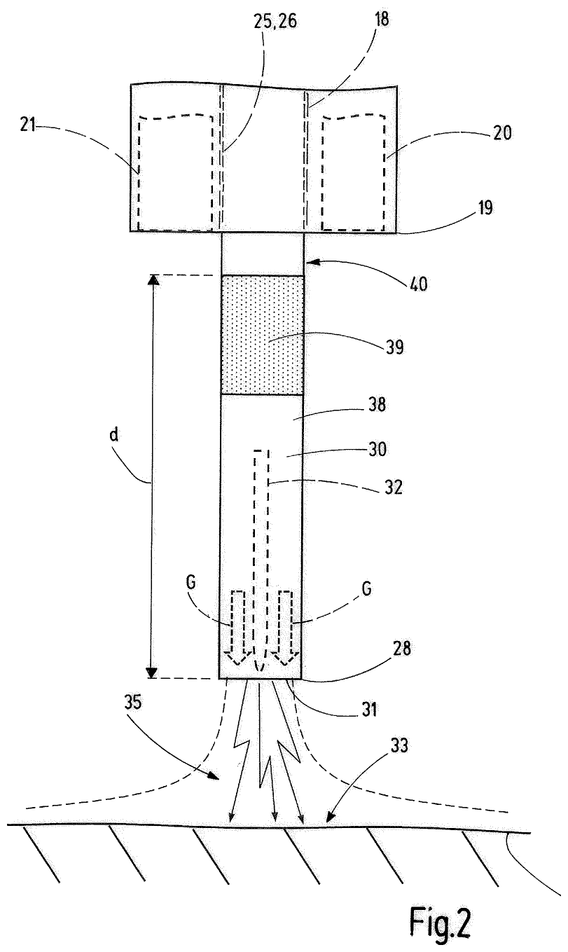

In the configuration of the probe 17 for plasma coagulation a fluid channel 30 is formed inside the instrument body 25 or the instrument hose 26 (FIG. 2). By means of the supply and operating unit 29, a fluid G such as for example an inert gas, e.g. argon, can be introduced. The fluid G flows through the fluid channel 30 to an exit opening 31 in the area of the distal end 28 of the instrument hose 26.

The electrode 32 is arranged inside the instrument hose 26 and adjacent to the exit opening 31. As explained, the electrode 32 is electrically connectable with the supply and operating unit 29 such that a radio frequency AC-voltage can be applied to the electrode 32. By means of the radio frequency AC-voltage the fluid, e.g. an inert gas G, can be ionized and a plasma 35 can be formed. As highly schematically illustrated in FIG. 2, energy is transferred on the tissue 34 to be treated within an application area 33 by means of the plasma—and according to the example the argon plasma—in direction E of the applied electric field. Depending on the dosage, e.g. on the amount and/or the time-dependent progress of the radio frequency AC-voltage and/or the influence duration on the application area 33, a depth effect is achieved in the tissue 34 to be treated due to the energy introduction by means of the plasma. The dosage of the energy introduction in the tissue 34 to be treated is optimum, particularly if during the treatment about two thirds of the desired depth effect is achieved. Due to aftereffects within about 72 hours after the treatment, the depth effect increases about the still missing third.

By means of the probe 17, a thermal or electrosurgical treatment and according to the example, an argon plasma coagulation of tissue 34 to be treated can thus be carried out. The argon plasma coagulation is contactless. Thus, the probe 17 does not get in contact with the tissue 34 in the application area 33.

In an end section 38 of the instrument body 25 and according to the example, of the instrument hose 26, at least one color mark 39 is present adjacent to its distal end 28, wherein the color mark 39 has a color with a defined color shade and/or a defined color saturation and/or a defined brightness. The color shade can be, e.g. a brown color shade. The color of the color mark 39 corresponds to the color that the treated tissue 34 adopts in the application area 33, if the energy introduced by the selected dosage results in the desired depth effect in the tissue 34. Therefore, the color of the treated tissue 34 in the application area 33 is an indicator for the achieved depth effect and the dosage, particularly the energy introduced per unit area. Based on the color mark 39, an optical comparison between the color of the color mark and the color of the tissue 34 in the application area 33 can be carried out after the argon plasma coagulation. Then it can be recognized whether the dosage has been correctly selected.

According to the example, the color of the color mark 39 is brown or brown-beige. In the embodiment the color of the at least one color mark 39 is the RAL-color with the number 1011.

The one color mark 39 or at least one of multiple color marks 39 comprise one or more continuous colored areas. One single continuous colored area is sufficient.

The one color mark 39 or at least one of multiple color marks 39 can have, as an option, also a texture and/or surface structure and/or frosted surface. In doing so, the real tissue structure can be represented better and the comparison between the textured color mark 39 and the color and/or structure of the tissue 34 can be simplified. In addition, a rough and/or frosted surface of the color mark 39 can avoid reflections, whereby the color comparison can be simplified.

As far as the one color mark 39 or at least one of multiple color marks 39 comprises one or more textures, they can be realized following VDI 3400 (surface reference sample measurement) according to the VDI-classes 33-42 with a maximum depth of roughness Rmax=18-49 μm and an arithmetic average roughness value Ra=4.5-12.5 μm, particularly VDI-class 36, Rmax=25 μm, Ra=6.3 μm.

In the embodiment at least one color mark 39 is provided on the shell surface 40 or the circumferential surface of the instrument hose 26 in the end section 38, e.g. by means of a printing process. For example, the color mark 39 can be a ring area that surrounds the shell surface 40 partly and preferably completely. In extension direction of the instrument hose 26 or the end section 38 the at least one color mark 39 comprises a length of at least 2 mm or at least 3 mm. Preferably the length of a color mark 39 in extension direction of the end section 38 is not larger than 5 mm or 6 mm or 7 mm or 10 mm.

For a surgeon additional markings can be provided on the instrument hose 26 and particularly on the shell surface 40 that can be configured in arbitrary colors and shapes.

Instead of a ring shape the at least one color mark 39 can also have any other arbitrary configuration. For example, the at least one color mark 39 can comprise at least one symbol and/or at least one sign (digit or letter) and/or at least one geometric figure. Thus, also a company lettering and/or a company logo can be used as color mark 39. FIG. 6a schematically illustrates a print of a color mark 39 in the form of symbols.

In the end section 38 of a further embodiment of the probe 17 illustrated in FIG. 6b, at least one of the color marks 39 is arranged, e.g. printed, directly adjoining the distal end 28 on the shell surface 40. This color mark can be completely or partly formed by a hollow cylindrical end piece 41. One or more additional ring-shaped color marks can be provided as an option that are, for example, arranged with different distances to the distal end 28.

In the embodiment of the probe 17 according to FIGS. 2, 3 and 6a, the one color mark 39 or all of the color marks 39 are arranged with distance to the distal end 28 of the instrument hose 26. Preferably the instrument hose 26 has a color apart from the at least one color mark 39 that is optically in contrast to the surrounding tissue, such that the instrument hose 26 can be recognized well during surgery and according to the example, the endoscopic intervention. Due to the distance of the at least one color mark 39 from the distal end 28, the exit opening 31 can be positioned in the desired distance from the application area 33 of the tissue 34.

By means of the at least one color mark 39 or—provided that multiple color marks 39 are present—one of the present color marks 39, a minimum distance d from the distal end 28 of the instrument hose 26 can be marked. For example, an edge of the color mark 39, particularly the edge of the color mark 39 opposite the distal end 28, can be arranged at a position of the end section 38 of the instrument hose 26 that defines the minimum distance d to the distal end 28. During the endoscopy the surgeon can recognize whether the probe 17 or its end section 38 is pushed out sufficiently far out of the operation channel 18 of the endoscope 15 in order to not damage the endoscope 15 and particularly the optics during treatment of the tissue 34.

In all embodiments of the instrument or the probe 17 the color mark 39 or one of the color marks 39 can be arranged directly adjacent to the distal end of the instrument body 25. Alternatively, the at least one color mark 39 can have a distance to the distal end 28. Both variations have their own advantages.

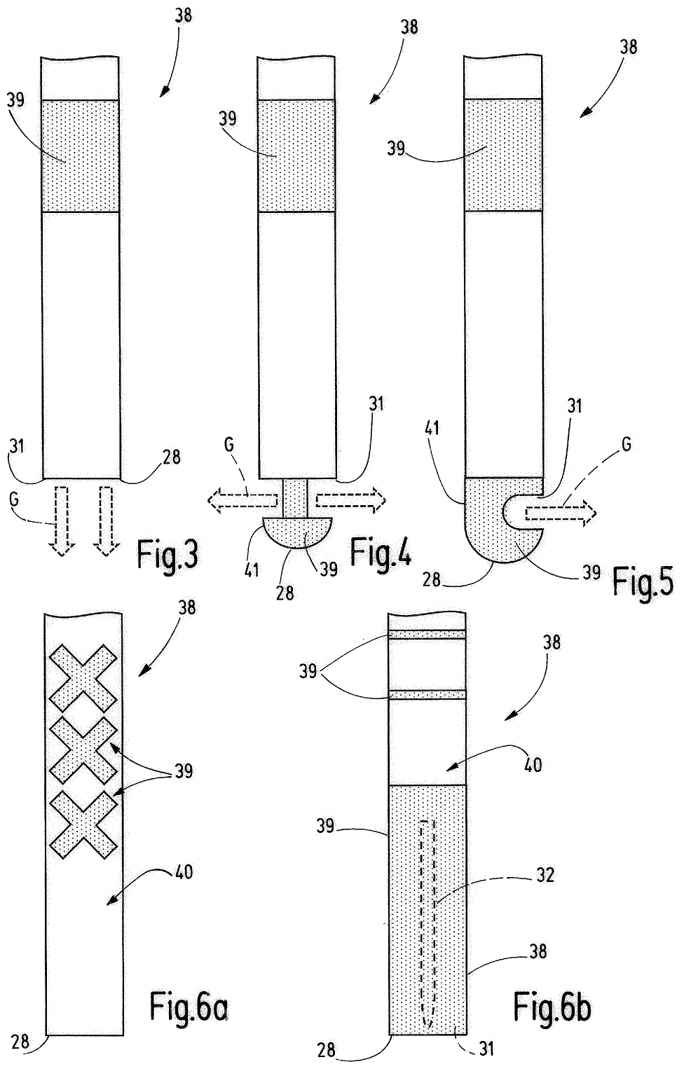

In FIGS. 3-5, 11 and 12 different configurations of the instrument body 25 or the instrument hose 26 are illustrated. The embodiment according to FIG. 3 corresponds to the embodiment shown in FIG. 2, in which the fluid G or the plasma is ejected in extension to the end section 38. Different thereto the instrument body 25 and according to the example, the instrument hose 26 comprises an end piece 41 in the embodiments according to FIGS. 4 and 5 that comprises the exit opening 31. By means of the end piece 41, the direction of the exiting fluid G or plasma can be varied. In the embodiment according to FIG. 4, the fluid G or the plasma exits substantially orthogonal to the extension direction of the end section 38 in all directions. In contrast thereto the exit opening 31 of the end piece 41 of FIG. 5 is configured such that the inert gas G or the plasma is ejected selectively laterally in one direction substantially orthogonal to the extension direction of the end section 38.

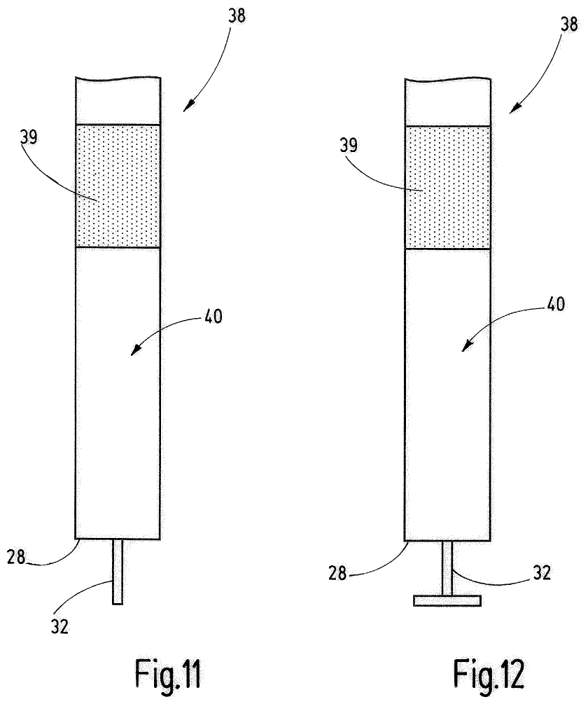

FIGS. 11 and 12 schematically show embodiments of the probe in which the electrode 32 projects from the distal end 28 of the instrument body 25. These probes can be configured without fluid channel 30. The electrode 32 can be brought in direct contact with the tissue 34 during the thermal treatment or influence the tissue 34 by spark creation without additional medium.

In the embodiments in which an end piece 41 is provided, the end piece 41 can form a color mark 39. For this it can be colored in the defined brown color of the color mark 39 or can be coated completely or partly. For example, the end piece 41 can be made of a ceramic material.

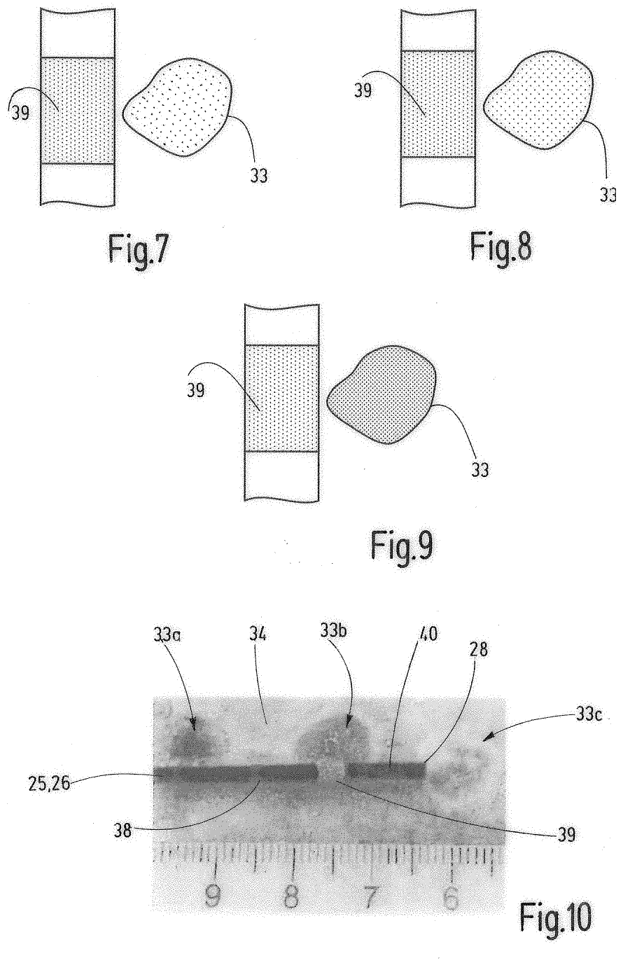

Based on FIG. 7-10, the function of the at least one color mark 39 is schematically explained. By positioning of the at least one color mark 39 directly adjacent to an application area 33 in which a tissue 34 has already been treated, a color comparison can be carried out. The brightness of the color is schematically illustrated in FIGS. 7-9 by the density of the dots.

It is apparent from FIG. 7 that the color of the color mark 39 is darker than the color of the tissue 34 in the application area 33. An underdosage exists and the tissue has to be further treated in the application area 33 in order to achieve the desired depth effect.

In contrast the color of the tissue 34 in the application area 33 according to FIG. 9 is darker than the color of the at least one color mark 39, such that an overdosage exists. The tissue 34 has been damaged or influenced in the application area 33 beyond the desired depth effect. The surgeon can recognize this and estimate the consequences or initiate necessary measures, if applicable.

FIG. 8 schematically illustrates the situation in which the color of the color mark 39 and the color of the treated tissue in the application area 33 correspond approximately and the surgeon recognizes that the dosage for achieving the desired depth effect in the tissue 34 directly after the treatment is correct (about two third of the depth effect achieved under consideration of the aftereffect).

The situations illustrated schematically in FIGS. 7-9 are again illustrated based on the photographic picture in FIG. 10. On the left side in the picture a first application area 33a can be seen, the color of which is darker than the color of the color mark 39. An overdosage exists. The color in the second application area 33b corresponds substantially to the color of the at least one color mark 39. In the second application area 33b the correct dosage of the introduced energy has been achieved. In a third application area 33c comprising a clearly brighter color than the color of the color mark 39, the energy introduction has been underdosed. By a further treatment of this third application area 33c, the desired dosage or depth effect can still be reached.

The invention refers to an instrument for thermal treatment of tissue 34, particularly for electrosurgical treatment and particularly for argon plasma coagulation. The instrument has an instrument body 25 extending between a proximal end 27 and a distal end 28. In an end section 38 adjoining a distal end 28 the instrument body 25 comprises at least one color mark 39 in a defined color. This color corresponds to the color of a treated tissue 34 that is created, if the dosage of an energy introduction for achieving a desired depth effect in the tissue 34 has been selected correctly. Preferably the instrument is configured as probe 17, particularly as endoscope probe and has a flexible bendable instrument body 25 that can be referenced as instrument hose 26.

### LIST OF REFERENCE SIGNS

15 endoscope
16 operating part
17 probe
18 operation channel
19 distal endoscope end
20 light source
21 objective lens
25 instrument body
26 instrument hose
27 proximal end of instrument hose
28 distal end of instrument hose
29 supply and operating unit
30 fluid channel
31 exit opening
32 electrode
33 application area
33a first application area
33b second application area
33c third application area
34 tissue
35 plasma
38 end section
39 color mark
40 shell surface
41 end piece
d minimum distance
E direction of electric field
G fluid

1. An instrument for thermal treatment of tissue (34), comprising:
an instrument body (25) that extends from a proximal end (27) to a distal end (28),
an electrode (32) configured for application of an electric voltage,
wherein in an area of the distal end (28) at least one color mark (39) is arranged on the instrument body (25), wherein a color of the color mark (39) is selected such that it corresponds to a color of a tissue (34) treated with a desired dosage and/or depth effect.

2. The instrument according to claim 1, wherein the color of the at least one color mark (39) is brown or brown-beige.

3. The instrument according to claim 1, wherein the color of the at least one color mark (39) is a RAL-color.

4. The instrument according to claim 1, wherein the at least one color mark (39) has a length in an extension direction of the instrument body (25) of at least 2 mm.

5. The instrument according to claim 1, wherein the at least one color mark (39) is provided at a shell surface (40) of the instrument body (25).

6. The instrument according to claim 5, wherein at least a section of the at least one color mark (39) indicates a predetermined minimum distance (d) from the distal end (28).

7. The instrument according to claim 6, wherein an edge of the at least one color mark (39) that is furthest away from the distal end (28) indicates the predetermined minimum distance (d) from the distal end (28).

8. The instrument according to claim 5, wherein the at least one color mark (39) extends in a circumferential direction about at least 50% of a circumference of the instrument body (25).

9. The instrument according to claim 8, wherein the at least one color mark (39) extends completely around the circumference of the instrument body (25).

10. The instrument according to claim 1, wherein the at least one color mark (39) is spaced from an exit opening (31).

11. The instrument according to claim 1, wherein the at least one color mark (39) is provided on an end piece (41) of the instrument body (25), wherein the end piece (41) includes an exit opening (31).

12. The instrument according to claim 1, wherein the instrument body (25) is configured as an instrument hose (26).

13. The instrument according to claim 12, wherein the instrument hose (26) is configured to be guided through an operation channel (18) of an endoscope (15).

14. The instrument according to claim 1, wherein the instrument is configured for plasma treatment of tissue (34).

15. The instrument according to claim 1, wherein a fluid channel (30) is provided in the instrument body (25) that is fluidically connected with an exit opening (31) in an area of the distal end (28), whereby the electrode (32) is arranged inside the instrument body (25) adjacent to the exit opening (31).

16. The instrument according to claim 1, wherein the desired dosage and/or depth effect corresponds to at least one of an amount of a radio frequency AC-voltage applied to the electrode, a time-dependent progress of the radio frequency AC-voltage applied to the electrode, and a thermal influence duration on the tissue.

17. The instrument according to claim 1, wherein the at least one color mark is visible from an exterior of the instrument body.

18. The instrument according to claim 1, wherein the color of the at least one color mark is equivalent to a color identified by one of RAL-numbers 1001, 1005, 1011, 1024, 8001, 8003, 8007 or 8011.

19. The instrument according to claim 1, wherein the instrument body has at least one other color different from the color of the at least one color mark.

20. The instrument according to claim 19, wherein the at least one other color of the instrument body adjacent to the at least one color mark is darker than the color of the at least one color mark.
